(19) United States
(12) Patent Application Publication
(10) Pub. No.: US 2010/0041986 A1
(43) Pub. Date: Feb. 18, 2010

(54) ABLATION AND MONITORING SYSTEM INCLUDING A FIBER OPTIC IMAGING CATHETER AND AN OPTICAL COHERENCE TOMOGRAPHY SYSTEM

(76) Inventors: Tho Hoang NGUYEN, Huntington Beach, CA (US); Peter C. CHEN, Irvine, CA (US); Alan DE LA RAMA, Cerritos, CA (US); Yu LIU, Irvine, CA (US)

Correspondence Address: MATTINGLY & MALUR, P.C., 1800 DIAGONAL ROAD, SUITE 370, ALEXANDRIA, VA, 22314, US

(21) Appl. No.: 12/508,000

(22) Filed: Jul. 23, 2009

Related U.S. Application Data
(60) Provisional application No. 61/135,872, filed on Jul. 23, 2008.

(51) Int. Cl. A61B 6/02; A61B 18/14

(52) U.S. Cl. 600/427; 606/33

(57) ABSTRACT

An ablation and monitoring system comprises a catheter, an optical coherence tomography (OCT) system, and an ablation generator. The catheter comprises one or more optical fibers to transmit a light beam to a tissue material and collect a reflected light from the tissue material. The OCT system is in optical communication with the catheter via the one or more optical fibers, providing the light beam to the one or more optical fibers and receiving the reflected light from the one or more optical fibers. The ablation generator is in electrical communication with the OCT system and with the catheter. The ablation generator provides radio frequency energy to the catheter for ablating the tissue material, monitors and assesses the ablation based on an information signal received from the OCT system.

### CROSS-REFERENCE TO RELATED APPLICATIONS

This application claims the benefit of U.S. Provisional Application Ser. No. 61/135,872, filed on Jul. 23, 2008, entitled “Ablation and monitoring system including a fiber optic imaging catheter and an optical coherence tomography system”, which is hereby incorporated by reference in its entirety.

### BACKGROUND OF THE INVENTION

This invention relates generally to ablation systems and catheter devices, and more specifically to ablation systems with monitoring and evaluation capabilities.

Catheters are flexible, tubular devices that are widely used by physicians performing medical procedures to gain access into interior regions of the body. Certain types of catheters are commonly referred to as irrigated catheters that deliver fluid to a target site in an interior region of the body. Such irrigated catheters may deliver various types of fluid to the patient, including, for example, medications, therapeutic fluids, and even cooling fluids for certain procedures wherein heat is generated within targeted areas of the body.

For example, ablation catheters are sometimes used to perform ablation procedures to treat certain conditions of a patient. A patient experiencing arrhythmia, for example, may benefit from ablation to prevent irregular heart beats caused by arrhythmogenic electrical signals generated in cardiac tissues. By ablating or altering cardiac tissues that generate such unintended electrical signals the irregular heart beats may be stopped. Ablation catheters may include one or more ablation electrodes supplying radiofrequency (RF) energy to targeted tissue. With the aid of sensing and mapping tools, an electro-physiologist can determine a region of tissue in the body, such as cardiac tissue, that may benefit from ablation.

Once a tissue is targeted for ablation, a catheter tip having one or more ablation electrodes may be positioned over the targeted tissue. The ablation electrodes may deliver RF energy, for example, supplied from a generator, to create sufficient heat to damage the targeted tissue. By damaging and scarring the targeted tissue, aberrant electrical signal generation or transmission may be interrupted. In some instances irrigation features may be provided in ablation catheters to supply cooling fluid in the vicinity of the ablation electrodes to prevent overheating of tissue and/or the ablation electrodes.

Existing ablation catheters do not have fiber optic imaging capability to provide a physician with real-time assessment of the targeted tissue, tissue contact with the catheter tip, depth and volume of lesion, and other information.

Existing ablation systems do not have information inputs that are derived from optical signals from an ablation catheter that has fiber optic imaging capability to better monitor, assess and control the ablation process in real time.

### BRIEF SUMMARY OF THE INVENTION

An ablation and monitoring system comprises a catheter, an optical coherence tomography (OCT) system, and an ablation generator. The catheter comprises one or more optical fibers to transmit a light beam to a tissue material and collect a reflected light from the tissue material. The OCT system is in optical communication with the catheter via the one or more optical fibers, providing the light beam to the one or more optical fibers and receiving the reflected light from the one or more optical fibers. The ablation generator is in electrical communication with the OCT system and with the catheter. The ablation generator provides radio frequency energy to the catheter for ablating the tissue material, monitors and assesses the ablation based on an information signal received from the OCT system.

### BRIEF DESCRIPTION OF THE DRAWINGS

FIG. 1 is a block diagram illustrating the system 100 of the present invention.

FIG. 2 illustrates an embodiment of the catheter 110.

FIG. 3 shows an external view of the distal region 240 of the catheter 110.

FIG. 4A shows a longitudinal cross sectional view of an embodiment of the distal region 240 of the catheter 110.

FIG. 4B shows an external view of an embodiment 400 of the distal region 240 of the catheter 110.

FIG. 4C shows a longitudinal cross sectional view of the embodiment 400 of the distal region 240 of the catheter 110.

FIG. 5 illustrates a common-path interferometer system 500 for OCT imaging.

FIG. 6 shows a diagram of an embodiment 600 of the OCT system 120, which is a five-channel OCT system using common-path interferometer.

### DETAILED DESCRIPTION OF THE INVENTION

An ablation and monitoring system comprises a catheter, an optical coherence tomography (OCT) system, and an ablation generator. The catheter comprises one or more optical fibers to transmit a light beam to a tissue material and collect a reflected light from the tissue material. The OCT system is in optical communication with the catheter via the one or more optical fibers, providing the light beam to the one or more optical fibers and receiving the reflected light from the one or more optical fibers. The ablation generator is in electrical communication with the OCT system and with the catheter. The ablation generator provides radio frequency energy to the catheter for ablating the tissue material, monitors and assesses the ablation based on an information signal received from the OCT system.

In one embodiment, the ablation and monitoring system also includes a fluid pump in fluid communication with the catheter and in electrical communication with the ablation generator. The fluid pump receives instructions from the ablation generator and provides fluid to the catheter to irrigate the catheter in accordance with the instructions.

The OCT system includes at least one common-path interferometer. In one embodiment, the OCT system is a multi-channel OCT system.

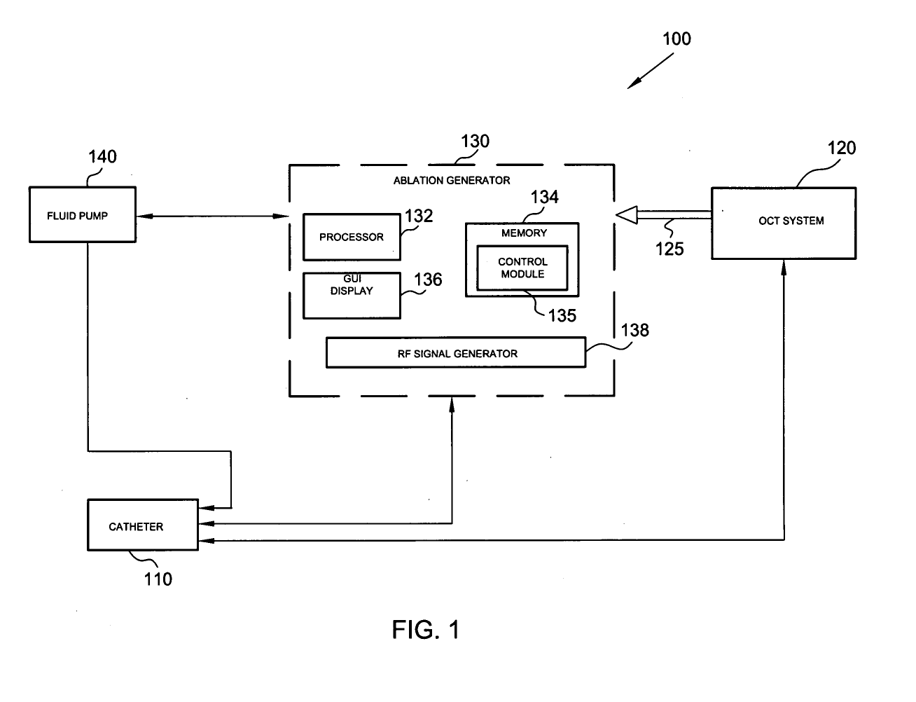

FIG. 1 is a block diagram illustrating the system 100 of the present invention. System 100 comprises a catheter 110, an optical coherence tomography (OCT) system 120, an ablation generator 130, and a fluid pump 140.

The catheter 110 of the present invention is an irrigated ablation catheter that also comprises optical fibers to transmit light to and collected reflected light from the tissue undergoing ablation. The catheter 110 is in optical communication with the OCT system 120, in electrical communication with the ablation generator 130, and in fluid communication with the fluid pump 140. The catheter 110 receives an optical signal from the OCT system 120 via one or more optical fibers. The optical fibers terminate at openings or transparent windows located in the distal portion of the catheter 110. The optical fibers are bi-directional. The optical fibers transmit the optical signals from the OCT system 120 through their ends into a tissue area and receive reflected optical signals which are sent back to the OCT system 120.

The ablation generator 130 comprises a processor 132, memory 134, a graphical user interface (GUI) 136, and a RF signal generator 138. The memory 134 includes a control module 135. The generator 130 receives the signal 125 from the OCT system 120. The image data from the signal 125 are displayed on the display of the GUI 136. The control module 135 processes information in the signal 125 to provide information including at least one of the following: lesion assessment (such as depth and volume of lesion), tissue contact assessment, signal change corresponding to tissue phase change, force sensing, thermal detection, tissue differentiation, and three-dimensional imaging. This information allows automatic or manual actions to be taken to prevent undesirable effects of ablation such as over-burning, formation of steam pop, etc. The information provided by the control module 135 is also displayed on the display of the GUI 136. The control module 135 also receives and processes user input received via the GUI 136.

The processor 132 executes instructions from the control module 135. In response to a user input requesting ablation, the control module 135 instructs the processor 132 to instruct the RF signal generator 138 to output an RF signal delivering RF energy for ablation to the catheter 110. The processor may also instruct the fluid pump 140 to pump fluid into the catheter 110 to irrigate it.

The OCT system 120 uses a reference optical signal identical to the optical signal originally transmitted to the catheter 110 to process the reflected optical signals into imaging and related information data signal 125, and sends the signal 125 to the ablation generator 130. In one embodiment, the OCT system 120 uses a frequency domain OCT technique that measures the magnitude and time delay of reflected light in order to construct depth profiles in the tissue being imaged. The OCT system 120 includes a high-speed swept laser, and a fiber-based Michelson interferometer with a photodetector. The OCT system 120 uses advanced data acquisition and digital processing techniques to enable real-time video rate OCT imaging. In one embodiment, the OCT system 120 employs common-path interferometers for OCT imaging. In a common-path interferometer, the reflection from the fiber end face is used as a reference beam. As such, the reference beam and reflection lights from an imaging object propagate in the same fiber. The common-path interferometer is very stable and substantially insensitive to the surrounding temperature, vibration, and even fiber bending or twisting. Stability of the interferometer is critical for OCT imaging in catheter applications during ablation in a heart cavity, with surrounding vibrations from the heart beating, the blood flowing, and with the pressure and temperature changing.

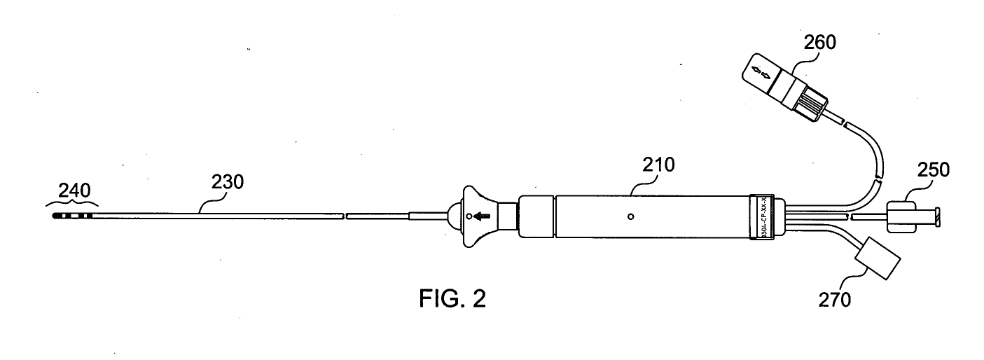

FIG. 2 illustrates an embodiment of the catheter 110. The catheter 110 comprises a control unit body 210, an elongated tubular catheter body 230 with a distal region 240, an irrigation port 250, a connector 260 to be connected to the ablation generator 130, and a fiber optic connector 270 to be connected to the OCT system 120.

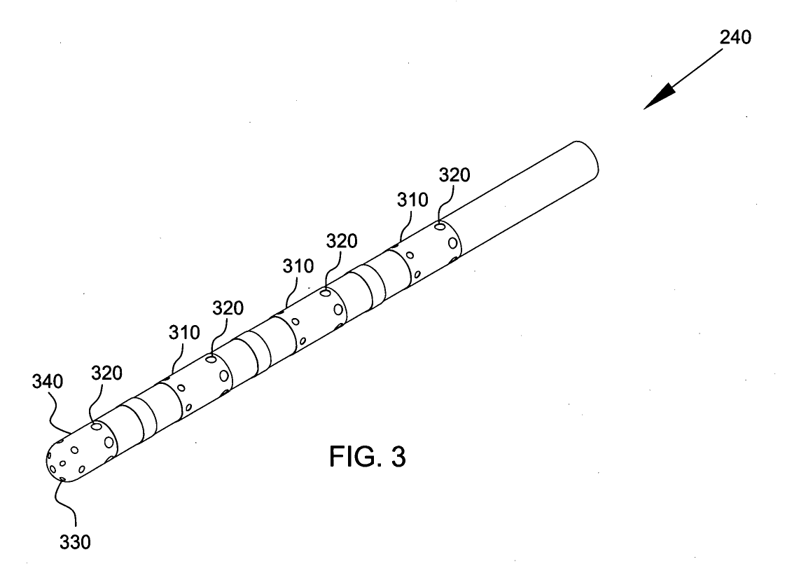

FIG. 3 shows an external view of the distal region 240 of the catheter 110. The catheter distal region 240 includes bands of electrodes 310 positioned spaced apart in different longitudinal sections on the catheter body. Each band of electrodes 310 further has a number of elution holes 320 for delivery of irrigation fluid from a main lumen formed in the catheter body to the exterior surface of the catheter. The catheter distal region 240 also includes one or more openings or transparent windows 330 to allow the terminating end of an optical fiber to transmit light and collect reflected light. A number of openings or transparent windows 330 may be located at various locations on the catheter distal region 240. At the terminal end of the distal region 240 is a catheter tip 340. In one embodiment, the catheter tip 340 includes at least one electrode and that electrode also includes a number of elution holes 320. The electrode at the distal end is referred to as the tip electrode. The catheter tip 340 may include at least one opening or transparent window 330.

The catheter tip 340 may be manufactured separately and attached to the rest of the elongated catheter body. The catheter tip 340 may be fabricated from suitable biocompatible materials to conduct ablation energy, such as RF energy, and to withstand temperature extremes. Suitable materials for the catheter tip include, for example, natural and synthetic polymers, various metals and metal alloys, naturally occurring materials, textile fibers, glass and ceramic materials, sol-gel materials, and combinations thereof. In an exemplary embodiment, the catheter tip 340 is fabricated from a material including 90% platinum and 10% iridium.

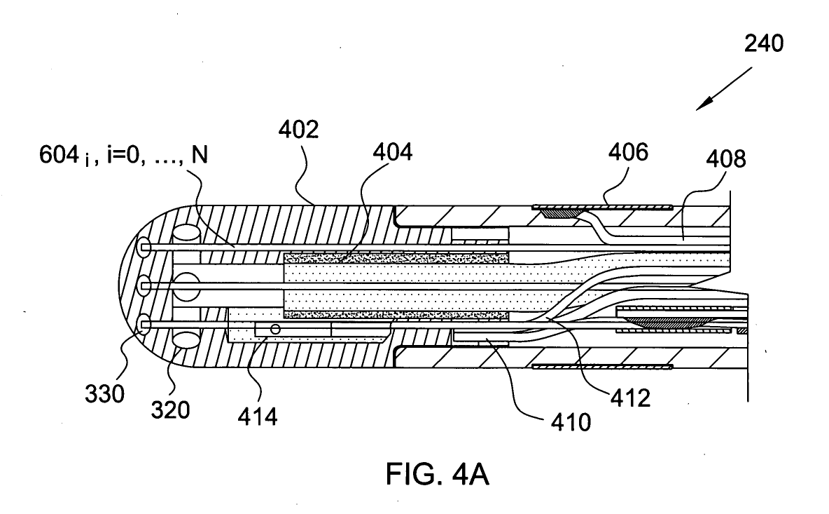

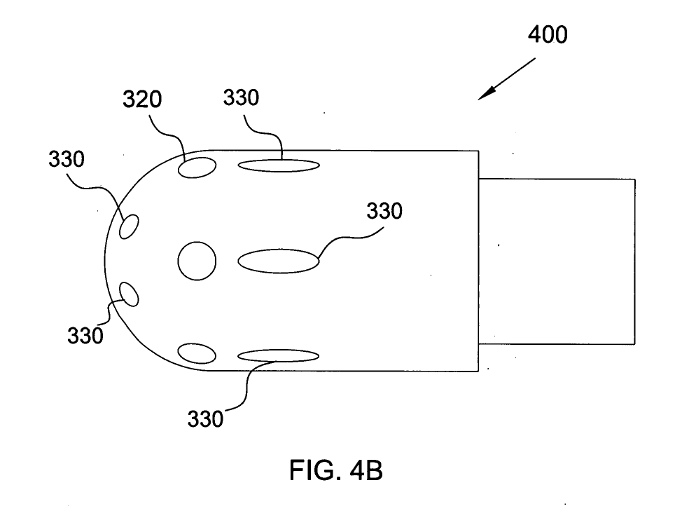

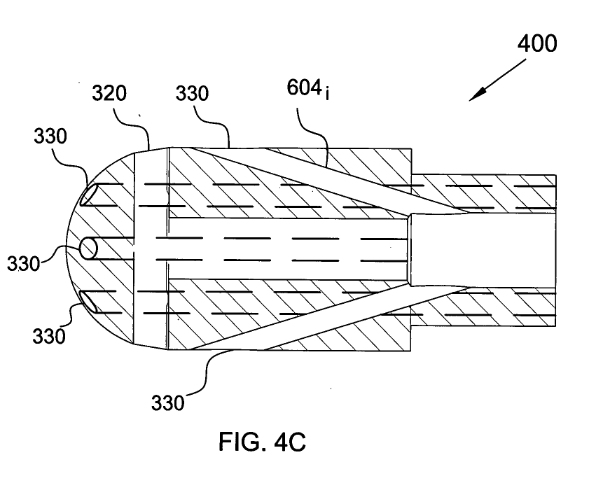

FIG. 4A shows a longitudinal cross sectional view of an embodiment of the distal region 240 of the catheter 110. In this embodiment, the distal region of the catheter 110 includes a tip electrode 402, a fluid lumen 404 for irrigating fluid to elution holes 320, a band electrode 406 connected to a band conductor wire 408, a tip conductor wire 410 connected to the tip electrode 402, a pull wire 412 for steering the distal region 240, a temperature sensor 414, and a plurality of optical fibers 604ᵢ, i=0, . . . , N, terminating at a plurality of openings or transparent windows 330.

FIG. 4B shows an external view of an embodiment 400 of the distal region 240 of the catheter 110. This embodiment 400 of the distal region 240 has a plurality of openings or transparent windows 330 placed at various locations.

FIG. 4C shows a longitudinal cross sectional view of the embodiment 400 of the distal region 240 of the catheter 110 shown in FIG. 4B. For simplicity, only the optical fibers 604ᵢ terminating at openings or transparent windows 330 and the fluid lumen irrigating fluid to elution holes 320 are shown. FIG. 4C shows the hidden view (represented by broken lines) of three optical fibers placed axially and terminating at the openings or transparent windows 330 located at the distal end of the catheter 110, and two optical fibers each placed at an angle and terminating at an opening or transparent window 330 placed at a location proximal to the distal end of the catheter 110. This configuration allows the optical fibers to transmit light to and collect reflected light from the tissue material at different angles. This results in a large cross-sectional angle of view of the tissue. This cross-sectional angle of view may be approximately 90 degrees. This configuration provides multi-directional OCT imaging.

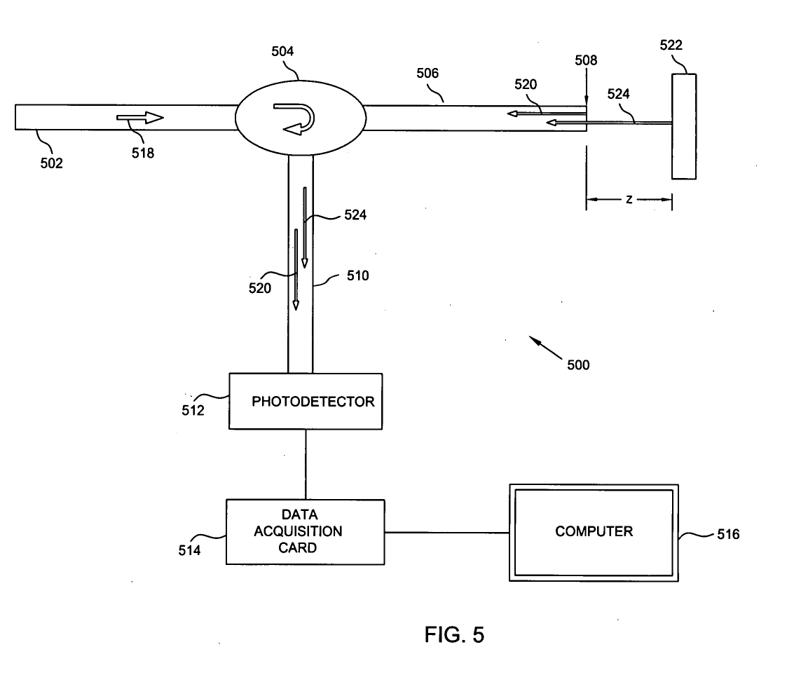

FIG. 5 illustrates a common-path interferometer system 500 for OCT imaging. System 500 comprises an optical fiber 502, an optical circulator 504, an optical fiber 506 having a fiber end face 508, an optical fiber 510, a photodetector 512, a data acquisition card 514, and a computer 516.

Referring to FIG. 5, a light beam 518 from a high-speed swept laser travels through optical fiber 502, then through the optical circulator 504 and through optical fiber 506, and illuminates an object 522 placed at a distance z from the fiber end face 508 of the optical fiber 506. The reflected light beam 520 from the fiber end face 508 is used as the reference beam. The reflected light beam 524 from the imaging object 522 and the reflected light beam 520 from the fiber end face 508 travel back in the same selected optical fiber 506 toward the optical circulator 504. The optical circulator 504 directs the object reflected light 524 and the reference beam 520 to travel to the photodetector 512. The photodetector 512 detects the interference signal which results from the interference between the reference beam 520 and the object reflected light 524, and outputs a corresponding analog electrical signal to the data acquisition card 514. The data acquisition card 514 receives the analog signal, processes it into proper format and sends the resulting information signal to the computer 516 for processing and display.

Optical scanning may be used to achieve a 2-dimensional or 3-dimensional imaging. When optical scanning is very difficult to implement or not economical, a fiber array or multi-channel OCT may be used to simulate the scanning to achieve a 2-dimensional or 3-dimensional imaging.

One way to control the strength of the reference beam to optimize the interference signal is to use angle-cleaved fibers. To reduce the reflection at the optical fiber end face 508 to about 1 percent, the tip of the optical fiber 506 may be angle-cleaved. It is noted that, when the optical fiber 506 is cleaved at 90 degrees, this results in a reflection of approximately 4 percent.

Another way to control the strength of the reference beam is to use Gradient-index (GRIN) fiber lens. GRIN fiber lens can be used to focus the laser beam to illuminate the imaging object and to collect more scattering lights from the imaging object to improve the signal-noise ratio (SNR). The length of GRIN lenses can be used to control the strength of the reference beam to optimize the interference signal, i.e., the OCT signal. Experiments showed that GRIN lenses provide a more controllable method for optimizing the interference signal than the method of angle-cleaved fibers.

With the common path interferometer system shown in FIG. 5, the intensity of the interference signal is expressed as:

$$I = {\vert {r}_{0} + {r}_{z} {e}^{j \frac{4 π \cdot z}{{λ}_{0} + Δλ sin ( 2 π {f}_{sweep} t )}} \vert}^{2} \qquad ( 1 )$$

where r₀ is the amplitude reflectance at the fiber end face, r_{z} is the amplitude reflectance at depth z of the imaging object, l₀ is the central wavelength, Dl is wavelength sweeping range, and f_{sweep} is the wavelength sweeping rate.

For simplicity, a top-hat spectral profile f(dl) is used to only consider the intensity I within the range of the spectral profile f(dl):

$$f ( δλ ) = \{ \begin{matrix}1 & \vert δλ \vert \leq {Δλ}_{fwhm} / 2 \\ 0 & \vert δλ \vert > Δ {λ}_{fwhw} / 2\end{matrix} \qquad ( 2 )$$

where Dl_{fwhm} is the laser instantaneous linewidth.

Simplifying Eq. (1), and ignoring the DC component r₀²+r_{z}², the intensity of the interference signal can be expressed as:

$$I ∼ 2 {r}_{o} {r}_{z} cos [ \frac{4 π \cdot z}{{λ}_{0} + Δλsin ( 2 π {f}_{sweep} t )} ] \qquad ( 3 )$$

By applying a fast Fourier Transform (FFT) to Eq. (3), it can be derived that the Fourier frequency F is directly proportional to the depth z and the amplitude of the Fourier component at Fourier frequency F is proportional to the amplitude reflectance r_{z}. It is noted that the re-clocking operation to achieve an equidistant spacing in frequency is required for the data stream when it is captured in equidistant time spacing.

$$\{ \begin{matrix}z = \frac{{λ}_{0}^{2}}{2 Δλ} \cdot \frac{F}{{f}_{sweep}} \\ {r}_{z} ∝ A ( F )\end{matrix} \qquad ( 4 )$$

where F is the Fourier frequency, and Λ(F) is the amplitude of the Fourier component at Fourier frequency F.

The OCT system of the present invention provides monitoring and assessment of tissue contact. When the optical fiber 506 touches the imaging object 522, F=0. Equation (4) shows that the scattering from depth z can be explored by the Fourier frequency F and the amplitude A(F) of the Fourier component at Fourier frequency F.

The OCT system of the present invention provides imaging of the ablation area, lesion assessment, tissue differentiation, and three-dimensional imaging. When the tissue is ablated or charred, the light reflectance r_{z} or scattering coefficient will be increased. The strength of the Fourier components will be significantly increased accordingly. The changes of tissue shape cause the imaging pattern to change.

The OCT system of the present invention provides warning for steam pop. It is very important to avoid steam pop during ablation since the presence of steam pop indicates that the tissue is seriously damaged. Before the steam pop actually happens, there is a lot of micro-pops generated by the overheating. The micro-pops will significantly increase the light scattering and thus can be monitored by the strength of the Fourier components, i.e., OCT intensity. Experiments have shown that OCT intensity is very sensitive to the presence of micro-pops. When micro-pops are detected, a warning for a steam pop is generated, and the ablation generator 130 reduces its ablation power and beeps for attention.

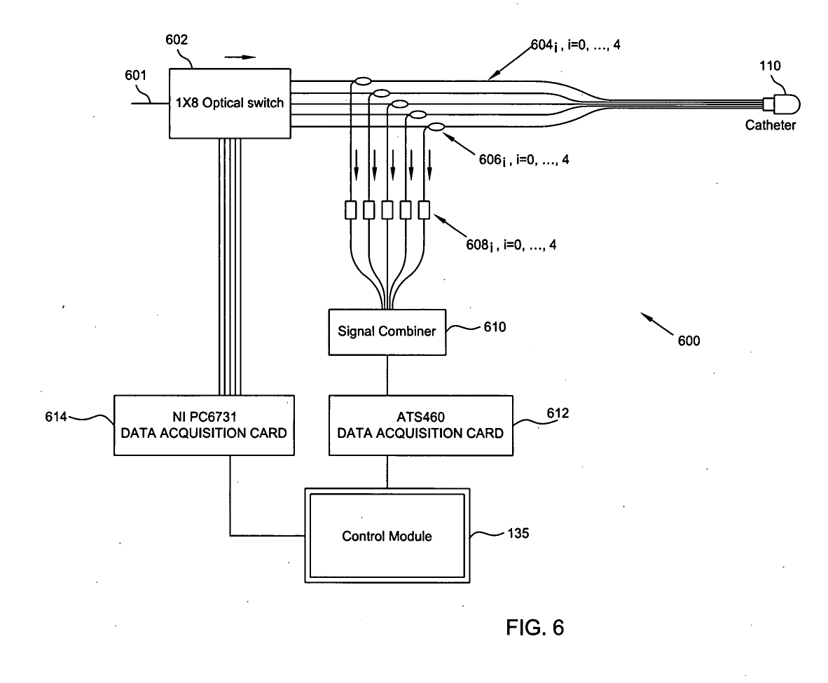

FIG. 6 shows a diagram of an embodiment 600 of the OCT system 120, which is a five-channel OCT system using common-path interferometer.

The OCT system 600 comprises an optical fiber 601, an optical switch 602, five optical fibers 604 which are connected via the fiber optic connector 270 (see FIG. 2) to five corresponding optical fibers which terminate inside the catheter 110, five optical circulators 606, five photo detectors 608, a signal combiner 610, a data acquisition card 612 which sends an analog information signal to the control module 135 of ablation generator 130. System 600 also includes a second data acquisition card 614 to send a digital control signal to the optical switch 602 to control the switch function. The data acquisition card 614 is in electrical communication with the control module 135. It is noted that this second data acquisition card 614 is not needed if the data acquisition card 612 can also output a digital control signal to the optical switch 602.

Referring to FIG. 6, a light beam from a high-speed swept laser travels through the optical fiber 601 and enters the optical switch 602 which, in accordance with the digital control signal received from the data acquisition card 614, selects one of the five optical fibers 604ᵢ, i=0, . . . ,4, and directs the light beam to the selected optical fiber 604ⱼ. The reflected light from an imaging object near the distal tip of the catheter 110 and the reflected light from the selected fiber end face, which is the reference beam, travel back in the same selected optical fiber toward the optical circulator 606ⱼ that is associated with the selected optical fiber 604ⱼ. The optical circulator 606ⱼ directs the object reflected light and the reference beam to travel to the associated photo detector 608ⱼ. The associated photo detector 608ⱼ detects the optical interference signal which results from the interference between the reference beam and the object reflected light, and outputs a corresponding analog electrical signal to the signal combiner 610. The one of the five optical fibers 604ⱼ, j=0, . . . ,4 combines the five analog signals received at its inputs into a single analog signal which is outputted to the data acquisition card 612. It is noted that, at any given time, due to the switching function of the optical switch 602, only one of the five analog signals has nonzero value. The data acquisition card 612 receives the analog signal, processes it into proper format and sends the resulting information signal to the control module 135 for processing as described above. The control module 135 may be included in the ablation generator 130 as shown in the system 100 of FIG. 1, or may be included in the OCT 120.

While the invention has been described in terms of several embodiments, those of ordinary skill in the art will recognize that the invention is not limited to the embodiments described, but can be practiced with modifications and alterations within the spirit and scope of the appended claims. The description is thus to be regarded as illustrative instead of limiting.

What is claimed is:

1. A system comprising:
a catheter comprising one or more optical fibers to transmit a light beam to a tissue material and collect a reflected light from the tissue material;
an optical coherence tomography (OCT) system in optical communication with the catheter via the one or more optical fibers, the OCT system providing the light beam to the one or more optical fibers and receiving the reflected light from the one or more optical fibers; and
an ablation generator in electrical communication with the OCT system and with the catheter, the ablation generator providing radio frequency energy to the catheter for ablating the tissue material, and monitoring and assessing the ablation based on an information signal received from the OCT system.

2. The system of claim 1 further comprises:
a fluid pump in fluid communication with the catheter and in electrical communication with the ablation generator, the fluid pump receiving instructions from the ablation generator and providing fluid to the catheter to irrigate.

3. The system of claim 1 wherein the OCT system comprises at least one common-path interferometer.

4. The system of claim 1 wherein the OCT system is a multi-channel OCT system.

5. The system of claim 1 wherein the one or more optical fibers are bidirectional.

6. The system of claim 4 wherein the ablation generator comprises:
a processor;
a memory coupled to the processor, the memory including a control module;
a graphic user interface coupled to the processor and memory; and
a radio frequency signal generator coupled to the processor.

7. The system of claim 6 wherein the control module processes an information signal received from the OCT system to provide at least one of the following: lesion assessment, tissue contact assessment, detection of micro-pops, force sensing, thermal detection, tissue differentiation, two-dimensional imaging of ablation area, and three-dimensional imaging of ablation area.

8. The system of claim 7 wherein the ablation generator provides warning for steam pop when the control module provides detection of micro-pops.

9. A catheter comprising:
an elongated body having a distal end, a proximal end, and at least one fluid lumen extending longitudinally therein;
a plurality of ablation electrodes being disposed on a distal portion of the elongated body, the plurality of ablation electrodes including a tip electrode;
a plurality of elution holes being disposed adjacent to the plurality of electrodes, at least one of the elution holes being disposed on the tip electrode;
a plurality of ducts establishing fluid communication between the elution holes and the at least one fluid lumen; and
at least one optical fiber extending longitudinally in the elongated body and terminating at at least one opening or transparent window disposed on the tip electrode.

10. The catheter of claim 9 wherein the at least one optical fiber extends axially in the elongated body.

11. The catheter of claim 9 wherein the at least one optical fiber extends non-axially in the elongated body and terminating at an angle at the at least one opening or transparent window.
